(19) United States
(12) Patent Application Publication
(10) Pub. No.: US 2012/0101306 A1
(43) Pub. Date: Apr. 26, 2012

(54) PROCESS FOR THE PREPARATION OF 1-METHYLCYCLOPENTANE DERIVATIVES

(75) Inventors: Andreas LANVER, Mannheim (DE); Klaus Ebel, Lampertheim (DE); Rainer Klopsch, Worms (DE); Werner Bertleff, Viernheim (DE); Richard Dehn, Ludwigshafen (DE); Joaquim Henrique Teles, Waldsee (DE); Helmut Kronemayer, Heidelberg (DE); Marcus Georg Schrems, Ludwigshafen (DE)

(73) Assignee: BASF SE, Ludwigshafen (DE)

(21) Appl. No.: 13/278,688

(22) Filed: Oct. 21, 2011

Related U.S. Application Data
(60) Provisional application No. 61/406,183, filed on Oct. 25, 2010.

(51) Int. Cl. C07C 41/14; C07C 1/20; C07C 5/29; C07C 29/04

(52) U.S. Cl. 568/579; 568/838; 585/639; 585/671

(57) ABSTRACT

Process for the preparation of 1-methylcyclopentene by thermal reaction of cyclohexanol or cyclohexene or mixtures of both compounds to give 1-methylcyclopentene, wherein the resulting by-products 3-methylcyclopentene and 4-methylcyclopentene (double-bond isomers of 1-methylcyclopentene) are returned to the reaction.

The present application includes, by reference, the prior U.S. Application 61/406,183 submitted on Oct. 25, 2010.

The present invention relates to a process for the preparation of 1-methylcyclopentene by thermal reaction of cyclohexanol or cyclohexene or mixtures of both compounds to give 1-methylcyclopentene, wherein the resulting by-products 3-methylcyclopentene and 4-methylcyclopentene (double-bond isomers of 1-methylcyclopentene) are returned to the reaction.

The present application likewise relates to a process for the preparation of cyclopentane derivatives of the following formula I

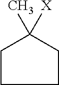

in which X is a hydroxyl group, an alkoxy group or a chlorine atom, by thermal reaction of cyclohexanol or cyclohexene to 1-methylcyclopentene (1^{st} stage) and subsequent addition of compounds HX, where X has the above meaning, onto the double bond of 1-methylcyclopentene (2^{nd} stage), wherein the resulting by-products 3-methylcyclopentene and 4-methylcyclopentene from the product mixture of the 1^{st} or 2^{nd} stage are returned to the reaction in the 1^{st} stage.

Cyclopentane derivatives of the above formula I are of importance as starting materials for chemical syntheses of a very wide variety of compounds.

It is known from U.S. Pat. No. 5,498,802 to prepare 1-methyl-1-hydroxycyclopentane in three separate reaction steps, with cyclohexanol being dehydrated to give cyclohexene in the first step, cyclohexene being isomerized to give 1-methylcyclopentene in the 2^{nd} step, and water being added on to 1-methylcyclopentene in the presence of isopropanol as solvent in the third step.

According to example 1 of U.S. Pat. No. 5,498,802, cyclohexanol dissolved in methanol is dehydrated at 250° C. over silicon dioxide to give cyclohexene. In the event of complete cyclohexanol conversion, a cyclohexene yield in the first step of 97.5% was achieved.

In the second step, cyclohexene is isomerized at 400° C. in the gas phase over silicon dioxide to give 1-methylcyclopentene. The 1-methylcyclopentene yield was 60.3% (example 2).

In the third step, a mixture of pure 1-methylcyclopentene, isopropanol and water is passed at 80° C. over Amberlyst15 resin. The 1-methylcyclopentene conversion was 41.9%, the yield of 1-methyl-1-hydroxycyclopentane 22.9% (example 3).

A disadvantage of the process according to U.S. Pat. No. 5,498,802 is the number of reaction steps and the low overall yield of 1-methyl-1-hydroxycyclopentane of only 13%, calculated over all part-steps. For the catalytic addition reaction of water onto 1-methylcyclopentene to give 1-methyl-1-hydroxycyclopentane pure 1-methylcyclopentene is used.

Neftekhimiya (1991), 31 (3), pages 386 to 390 (No. 12) describes the conversion of cyclohexanol to 1-methylcyclopentene in one step. Cyclohexanol is converted to methylcyclopentenene at 450° C. in the gas phase over chlorine-doped aluminum oxide.

U.S. Pat. No. 4,661,639 relates to a process for the preparation of cyclic alcohols by addition of water onto cyclic olefins using modified aluminum silicate catalysts.

Compared with the above prior art, it was the object of the present invention to provide a process for the preparation of cyclopentane derivatives of the formula I which can be carried out continuously, requires the fewest possible process steps and in which high yields and selectivity of the products are achieved in all process stages. In this connection, it was also an object of the invention to provide an improved process for the preparation of 1-methylcyclopentene. 1-Methylcyclopentene is required as starting material, or intermediate, for the preparation of cyclopentane derivatives; moreover, 1-methylcyclopentene is also, however, a starting material for other syntheses.

Accordingly, the processes defined above have been found.

In this application, the following seven compounds are referred to as follows:

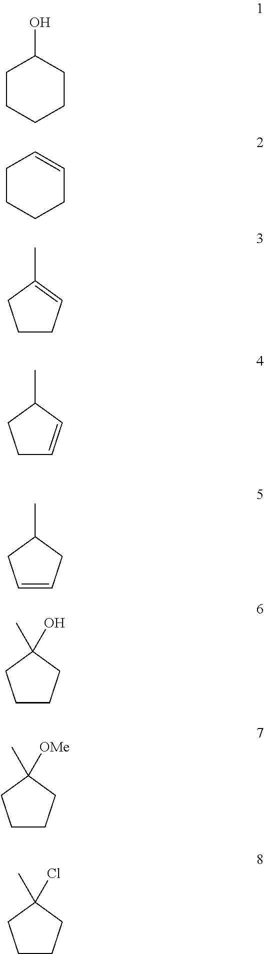

1: Cyclohexanol
2: Cyclohexene
3: 1-Methylcyclopentene
4: 3-Methylcyclopentene
5: 4-Methylcyclopentene
6: 1-Methyl-1-hydroxycyclopentane
7: 1-Methyl-1-methoxycyclopentane
8: 1-Methyl-1-chlorocyclopentane

The preparation of 1-methylcyclopentene

The statements below refer to the preparation of 1-methylcyclopentene as end product.

The starting compound of the process for the preparation of 1-methylcyclopentene is cyclohexanol or cyclohexene or a mixture of the two.

The reaction is a gas-phase reaction.

Starting from cyclohexene, a rearrangement to give 1-methylcyclopentene takes place; starting from cyclohexanol, an elimination of water to give cyclohexene takes place in the 1^{st} stage and then the subsequent rearrangement to 1-methylcyclopentene according to the following schematically represented reaction sequence:

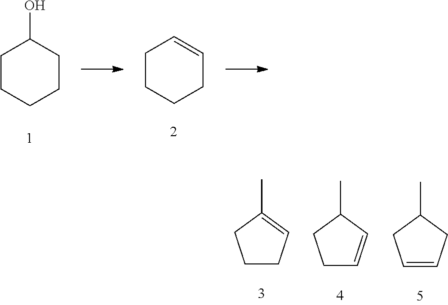

This gas-phase reaction is known per se.

Cyclohexene as starting compound can also be obtained by means of a separate dehydration of cyclohexanol carried out beforehand or any other desired process, e.g. by partial hydrogenation of benzene by the Asahi process.

A preferred starting compound is cyclohexanol or a mixture of cyclohexanol and cyclohexene, where the molar ratio of cyclohexanol to cyclohexene is 1:0.1 to 0.1:1.

The conversion of cyclohexanol to cyclohexene is generally complete.

The conversion can take place discontinuously or preferably continuously, i.e. with the continuous introduction of the starting materials and continuous discharge of the products.

In the process according to the invention, a recycling of the by-products 3-methylcyclopentene and 4-methylcyclopentene and also of any unreacted starting compounds takes place. The term “starting compounds” therefore comprises hereinbelow always also recycled compounds, e.g. recycled by-products and recycled, unreacted starting compounds, as is explained in more detail below.

The conversion preferably takes place in the gas phase in the presence of acidic catalysts. The gas-phase reaction can be carried out in reactors such as stirred reactors or tubular reactors.

The acidic catalysts can be arranged in the reactor as fixed bed or fluidized bed. A required inertization of the catalysts can be carried out with a carrier gas, such as e.g. nitrogen or argon.

Suitable solid acidic catalysts are e.g. SiO2, Al203, mixtures of SiO2 and Al203, alumosilicates, ZrO2, TiO2 or zeolites.

Suitable catalysts for the conversion of cyclohexanol or cyclohexene to 1-methylcyclopentene are also naturally occurring or synthetically produced zeolites.

The catalyst velocity is preferably 0.05 to 3, preferably 0.1 to 2, particularly preferably 0.2 to 1 kg of starting compounds per liter of catalyst and hour.

The residence time is in particular from 1 to 50 seconds, preferably 5 to 15 seconds.

The conversion can take place at temperatures of from 250 to 500, preferably 300 to 450, particularly preferably from 400 to 450° C.

The reaction pressure is not critical. It can be for example 0.1 to 10 bar, preferably 1 to 5 bar.

The product mixture obtained during the conversion comprises, besides the desired product 1-methylcyclopentene, also the following compounds:
the by-products 3-methylcyclopentene and 4-methylcyclopentene (double-bond isomers of 1-methylcyclopentene),
unreacted cyclohexene and
optionally water (if cyclohexanol has been used as starting material).

In general, during the conversion, 1 to 50 parts by weight, in particular 2 to 40 parts by weight, often 5 to 30 or 10 to 30 parts by weight, of 3-methylcyclopentene are formed per 100 parts by weight of 1-methylcyclopentene obtained.

In general, during the reaction, 1 to 50 parts by weight, in particular 1 to 30 parts by weight, often 1 to 20 or 2 to 20 parts by weight, of 4-methylcyclopentene are formed per 100 parts by weight of 1-methylcyclopentene obtained.

Preferably, the entire amount of 3- and 4-methylcyclopentene that is formed is returned to the reaction.

The gaseous reaction discharge is condensed. This can take place e.g. by adding an organic solvent such as toluene (quenching). When co-using cyclohexanol as starting material, the condensate consists of two liquid phases, one aqueous and one organic phase. The two phases are separated. The organic phase comprises the aforementioned product mixture, in particular the product of value 1-methylcyclopentene. The water phase is removed from the system.

The separation of the two liquid phases can take place by gravimetric phase separation. Suitable phase separation vessels are, for example, customary standard apparatuses and standard methods. Alternatively, water can be separated off from the condensed reaction discharge also by means of an azeotropic distillation.

The organic phase, which generally comprises unreacted cyclohexene (boiling point 83° C.), 1-methylcyclopentene (boiling point 76° C.), and also the two isomers 3-methylcyclopentene and 4-methylcyclopentene (boiling points 65-66° C.), can be worked up by distillation. The above boiling points are applicable for standard pressure; 1-methylcyclopentene can then be separated off from this organic phase and be used as end product in the desired manner.

Preferably, the entire residue of the organic phase, which comprises 3-methylcyclopentene and 4-methylcyclopentene and cyclohexene, is returned to the reaction.

The preparation of the cyclopentane derivatives of the formula I

### GENERAL

The process for the preparation of cyclopentane derivatives of the formula I is a two-stage process. In a 1^{st} stage, the thermal conversion of cyclohexanol or cyclohexene to 1-methylcyclopentene takes place; in the 2^{nd} stage, the addition of compounds HX takes place.

In formula I, X is a hydroxyl group, an alkoxy group, preferably a C1- to C10-alkoxy group or a chlorine atom. In particular, X is a hydroxyl group or an alkoxy group. Suitable alkoxy groups are in particular alkoxy groups having 1 to 10 carbon atoms, particularly preferably having 1 to 5 carbon atoms. Particularly preferably, X is an alkoxy group, in particular a C1- to C5-alkoxy group, e.g. a methoxy group, ethoxy group, isopropoxy group, n-propoxy group, n-butoxy group or a pentoxy group. X is very particularly preferably a methoxy group.

In the 1^{st} stage of the two-stage process, 1-methylcyclopentene is prepared. All of the above statements relating to the preparation of 1-methylcyclopentene are therefore applicable here accordingly, unless contradicted below.

During the subsequent reaction in the 2^{nd} stage, the addition of the compounds HX onto the double bond of 1-methylcyclopentene takes place.

In accordance with the desired radical X in formula I, HX is water (X=hydroxy group), HCl (X═Cl) or an alcohol which corresponds to the aforementioned alkoxy groups. Preferably, HX is accordingly water or preferably a C1- to 010-, in particular a C1- to C5-alcohol. Particular preference is given to methanol.

In the 2^{nd} stage, 1-methylcyclopentene is preferably reacted in the liquid phase in the presence of acidic catalysts with compounds HX to give cyclopentane derivatives of the formula I.

The reaction can be carried out at temperatures of for example 20 to 100° C., preferably 50 to 90° C., particularly preferably 40 to 90° C.

The reaction pressure is not critical. It can be for example 0.1 to 10 bar, preferably 1 to 5 bar.

The molar ratio of 1-methylcyclopentene to the compounds HX can be e.g. 1:10 to 10:1. In one preferred embodiment, HX is used in a molar excess; the molar ratio of 1-methylcyclopentene to the compounds HX is then in particular 1:1 to 1:10, particularly preferably 1:2 to 1:5.

The acidic catalysts used are preferably solid acidic catalysts, such as e.g. highly acidic ion exchangers or zeolites, as are also specified in U.S. Pat. No. 5,498,802.

The solid acidic catalysts can either be arranged in a fixed manner in a reactor or are suspended in the liquid phase.

The catalyst velocity can be for example 20 to 1, preferably 15 to 3, particularly preferably 5 to 3 kg, of methylcyclopentenes per liter of catalyst and hour.

The residence time can be for example 5 minutes to 2 hours, in particular 5 minutes to 2 hours and preferably 10 minutes to 30 minutes.

In the case of a hydration (HX═H₂O), the reaction in the 2^{nd} stage preferably takes place in the presence of a polar organic solvent, e.g. secondary alcohols such as e.g. sec-butanol and isopropanol. The solvent serves as solubility promoter between the water and the nonpolar starting material 1-methylcyclopentene.

The reaction discharge from stage 2 comprises the desired cyclopentane derivatives of the formula I, unreacted 1-methylcyclopentene, any isomers thereof as by-products, and any unreacted HX and optionally solvents. If the acidic catalyst was suspended in the reaction mixture, it can be separated off by filtration and returned to synthesis step 2.

The reaction discharge can be worked up by distillation.

For the purification and separation off of the desired cyclopentane derivative of the formula I, fractional distillations can be carried out.

If X in formula I is a C1- to C10 alkoxy group and if, in the 2^{nd} stage, accordingly the addition reaction of the C1- to C10 alcohol onto 1-methylcyclopentene takes place, in the 2^{nd} stage, in one preferred embodiment, a reactive distillation is carried out. In the case of reactive distillation, the reaction of the starting compounds takes place while carrying out the distillation.

In the present case, during the reactive distillation, the alcohol is added on to the double bond of the 1-methylcyclopentene, and the product or product mixture obtained as a result is separated off by distillation. The product or product mixture is produced here as bottom product on account of the boiling point difference between the resulting cyclopentane derivative of the formula I (preferably 1-methyl-1-methoxycyclopentane) and the starting materials. By means of a reactive distillation, high conversions are possible for equilibrium reactions since the product which forms is removed directly.

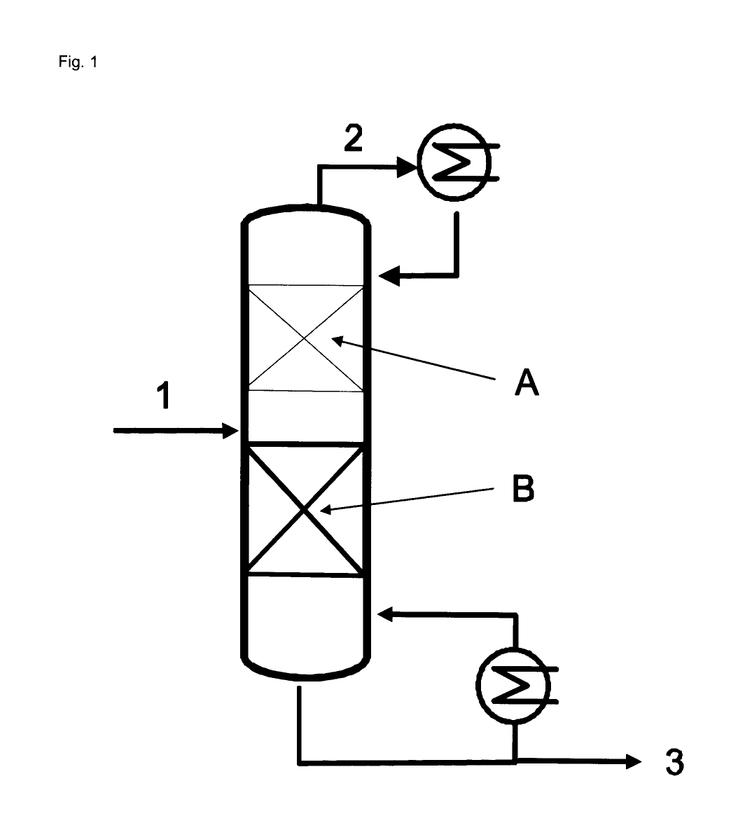

In a distillation apparatus suitable for continuous operation, as in FIG. 1, the starting materials (here C1 to C10 alcohol, preferably methanol, and 1-methylcyclopentene, and, where present, recycled by-products) can be introduced into the apparatus in gaseous or liquid form (stream 1 in FIG. 1). In the column, the conversion to the cyclopentane derivative of the formula I (preferably 1-methyl-1-methoxycyclopentane) takes place. The readily volatile starting compounds are condensed at the top of the column and returned to the column (stream 2). The less volatile cyclopentane derivative of the formula I (preferably 1-methyl-1-methoxycyclopentane) and less volatile by-products accumulate in the bottom of the column and can be drawn off (stream 3).

The distillation apparatus can preferably comprise customary internals for promoting distillative separation (packings or column trays) and the acidic catalyst, e.g. in the form of a fixed bed or fluidized bed (see internals A and B in FIG. 1).

The process in the 2^{nd} stage can be carried out discontinuously or continuously; preferably, the process in the 2^{nd} stage is carried out continuously.

Preferably, the overall process comprising the 1^{st} and 2^{nd} stage is therefore a process that is carried out continuously.

### The Recycle

An essential feature of the two-stage process according to the invention for the preparation of cyclopentane derivatives is that 3-methylcyclopentene and 4-methylcyclopentene, and any unreacted starting compounds in the product mixture from the 1^{st} or 2^{nd} stage are returned to the reaction if the 1^{st} stage.

Recycle from 1^{st} Stage

In one embodiment, 3-methylcyclopentene and 4-methylcyclopentene and any unreacted starting materials, i.e. cyclohexene, from the product mixture in the 1^{st} stage are returned to the reaction in the 1^{st} stage.

In this embodiment, therefore, 1-methylcyclopentene is separated off from the product mixture in the 1^{st} stage as described above and passed to the 2^{nd} stage, while 3-methylcyclopentene and 4-methylcyclopentene and preferably also the unreacted cyclohexene are returned to the reaction in the 1^{st} stage. In particular, as described above for the preparation of 1-methylcyclopentene, the entire organic residue after separating off the 1-methylcyclopentene is returned.

If cyclohexanol is used as starting compound alone or in a mixture with cyclohexene, the product mixture comprises water from the dehydration of cyclohexanol to cyclohexene. Water is produced as a separate aqueous phase and can easily be separated off from the organic phase, as is described above. The aqueous phase is discarded and not returned to the reaction in the 1^{st} stage.

In this embodiment, in addition to the recycle in the 1^{st} stage, a recycle can also take place in the 2^{nd} stage. In particular, unreacted starting materials from the 2^{nd} stage, i.e. 1-methylcyclopentene and unconsumed HX, are returned to the reaction in the 2^{nd} stage.

Recycle from Product Mixture of the 2^{nd} Stage

In a second embodiment of the two-stage process, after the 1^{st} stage, no separation off and recycle of 3-methylcyclopentene and 4-methylcyclopentene takes place. Instead, 3-methylcyclopentene and 4-methylcyclopentene from the product mixture in the 2^{nd} stage is returned to the reaction in the 1^{st} stage.

In this second embodiment, the product mixture from the 1^{st} stage is condensed and the organic phase, without separating off the by-products 3-methylcyclopentene and 4-methylcyclopentene and without separating off the unreacted starting compounds (cyclohexene), is fed to the further reaction in the 2^{nd} stage. If an aqueous phase is produced during the condensation of the product mixture on account of the (co-)use of cyclohexanol, this is preferably separated off and discarded if no water is required in stage 2. However, if an addition reaction of water is to take place in stage 2 (HX═H₂O), the aqueous phase is also fed to the 2^{nd} stage.

The product mixture obtained in the 2^{nd} stage accordingly comprises
the desired cyclopentane derivative of the formula I,
unreacted 1-methylcyclopentene and HX (starting materials for stage 2)
the by-products from stage 1: 3-methylcyclopentene and 4-methylcyclopentene
unreacted starting materials from stage 1: cyclohexene
optionally water

In the case of an addition reaction of water (HX═H₂O), preferably further water is added in the second stage. In addition to the water from the 1^{st} stage which is used in the 2^{nd} stage, an addition of from 1 to 100 mol, in particular 10 to 80 mol, of water per 1 mol of 1-methylcyclopentene is preferred.

From the product mixture of the 2^{nd} stage, the desired 1-methylcyclopentane derivative can be separated off from the aforementioned compounds by fractional distillation as high-boiling components and, if necessary, be purified again by distillation.

Preferably, unreacted HX, in particular water and alkanols, is also separated off from the product mixture in the 2^{nd} stage and not returned to the 1^{st} stage.

This then leaves as the organic residue from the 2^{nd} stage:
unreacted 1-methylcyclopentene (starting materials for stage 2)
the by-products from stage 1: 3-methylcyclopentene and 4-methylcyclopentene
unreacted starting materials from stage 1: cyclohexene

In this embodiment, these compounds are then returned to the reaction in the 1^{st} stage.

The process according to the invention for the preparation of 1-methylcyclopentene and also the two-stage process according to the invention for the preparation of cyclopentane derivatives are suitable in particular for a continuous process procedure. The yield of 1-methylcyclopentene or of desired cyclopentane derivative is high.

The recycling of 3-methylcyclopentene, 4-methylcyclopentene and cyclohexene does not lead to an increased formation of by-products.

In particular, it can be established that the isomers 3-methylcyclopentene and 4-methylcyclopentene rearrange to the desired compound 1-methylcyclopentene upon recycling to the 1^{st} stage and increase the yield.

Furthermore, it can be established that a presence of the isomers 3-methylcyclopentene and 4-methylcyclopentene in the 2^{nd} stage is not disadvantageous and no or barely any new by-products as a result of an addition of HX onto these isomers or else onto cyclohexene are observed.

The following working examples relating to stage 1 (Examples 1 to 3) and to stage 2 (Examples 6 to 14) show that the reactions take place with high selectivity. Example 5 shows the conversion, that takes place upon recycling, of the undesired by-products 3-methylcyclopentene and 4-methylcyclopentene to give the desired 1-methylcyclopentene. Example 14 shows that the presence of 3-methylcyclopentene, 4-methylcyclopentene and cyclohexene in the 2^{nd} stage does not lead to new by-products.

### EXAMPLES

The compounds below are referred to in the examples by the chemical name stated below or simply by the associated number.

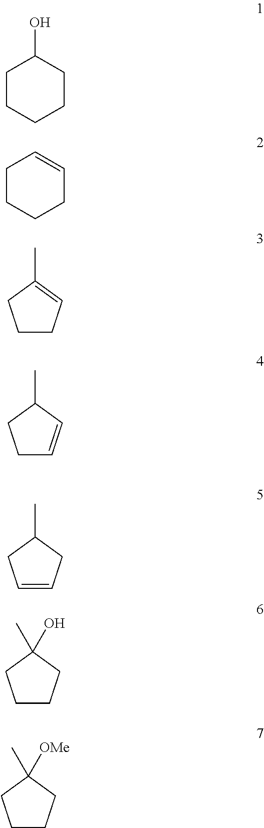

1: cyclohexanol
2: cyclohexene
3: 1-methylcyclopentene
4: 3-methylcyclopentene
5: 4-methylcyclopentene
6: 1-methyl-1-hydroxycyclopentane
7: 1-methyl-1-methoxycyclopentane

Abbreviations used: GC area %: gas chromatography area percent

### A) Gas-phase reaction of cyclohexanol to 1-methylcyclopentene

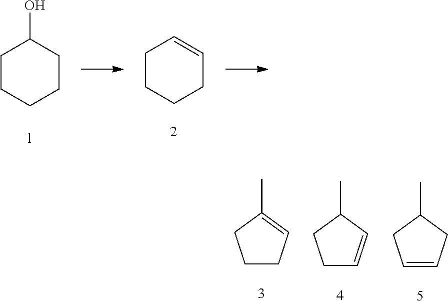

### Examples 1-3

A glass reactor (internal diameter 27 mm; height 500 mm) with electrical heating and temperature measurement was charged with a catalyst amount of ca. 300 ml. The starting material (1) was continuously introduced in trickle mode. In the bottom of the glass reactor, condensation was carried out in a 1 l flask with attached condenser using toluene as quenching liquid. The carrier gas used was nitrogen. The aqueous phase was separated off from the two-phase mixture in the separating funnel.

The organic phase was analyzed by GC.

| | | | | | Composition in | | | | |
|---|---|---|---|---|---|---|---|---|---|
| | | T | Carrier | Space | GC area % [a] | | | | |
| Example | Catalyst | [° C.] | gas N2 | velocity | 4 | 5 | 3 | 2 | 1 |
| 1 | SiO₂ | 400 | 5 l/h | 371 g/kg/h | 11 | 4 | 46 | 32 | 0 |
| 2 | SiO₂ + | 400 | 3 l/h | 112 g/kg/h | 10 | 4 | 54 | 28 | 0 |
| | 20% | | | | | | | | |
| | H₃PO₄ | | | | | | | | |
| 3 | SiO₂ + | 450 | 3 l/h | 117 g/kg/h | 11 | 4 | 58 | 20 | 0 |
| | 20% | | | | | | | | |
| | H₃PO₄ | | | | | | | | |

[a] Toluene deducted

### Example 4

In a column of height 120 cm containing 3×3 mm mesh rings (ca. 60 theoretical plates), a mixture consisting of the compounds 2, 3, 4 and 5 was subjected to discontinuous fractional distillation at atmospheric pressure. The top temperature was a constant 72° C. as compound 3 passed over.

| | | | | | | | Boiling |
|---|---|---|---|---|---|---|---|
| | | Compounds [in GC area %] | | | | | point |
| Sample | g | 4 | 5 | 3 | 2 | toluene | [° C.] |
| Feed | 724 | 8 | 3 | 33 | 40 | 13 | |
| Fr.1 | 50 | 68 | 25 | 3 | 0 | 0 | 63-66° C. |
| Fr.2 | 24 | 54 | 24 | 11 | 0 | 0 | |
| Fr.3 | 15 | 22 | 12 | 52 | 0 | 0 | |
| Fr.4 | 14 | 10 | 6 | 75 | 0 | 0 | |
| Fr.5 | 25 | 4 | 2 | 88 | 0 | 0 | 72° C. |
| Fr.6 | 21 | 1 | 1 | 96 | 0 | 0 | |
| Fr.7 | 21 | 0 | 0 | 98 | 0 | 0 | |
| Fr.8 | 37 | 0 | 0 | 99 | 0 | 0 | |
| Fr.9 | 34 | 0 | 0 | 99 | 1 | 0 | |
| Fr.10 | 30 | 0 | 0 | 97 | 2 | 0 | |
| Bottom | 393 | 0 | 0 | 2 | 68 | 27 | 83° C. |

This example shows the ability of compound 3 to be separated off by distillation from the resulting product mixture in the 1^{st} stage.

### Example 5

The same experimental set-up as in Examples 1-3 was used (catalyst: SiO₂+20% H₃PO₄). The feed substance used was the following mixture: compounds 3 (11 GC area %), 4 (56 GC area %), 5 (24 GC area %). At a temperature of 400° C., a carrier gas stream (N2) of 3 l/h and a catalyst space velocity of 146 g/kg/h, following condensation in toluene, the following mixture was obtained: compounds 3 (63 GC area %), 4 (12 GC area %), 5 (5 GC area %).

The experiment demonstrates that compounds 4 and 5 can be isomerized again into the desired product 3. Upon recycling these isomers, the yield is therefore increased accordingly.

### B) Hydration of 1-methylcyclopentene

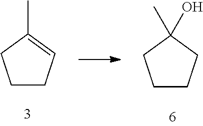

### Examples 6-9

In a continuously operated glass reactor (internal diameter 25 mm; height 200 mm), an ion exchanger (Amberlyst type) was introduced as catalyst. In trickle mode, at a constant internal volume, the mixture consisting of 1-methylcyclopentene, isopropanol (iPrOH) and water was introduced from a storage container via a membrane pump.

The product mixture was analyzed by GC. The yields and conversions were calculated from GC % by weight results.

| | Feed | | | | | | | | |
|---|---|---|---|---|---|---|---|---|---|
| | (3) | iPrOH | H₂O | | | Residence | Conversion | Yield | Selectivity |
| | [% by | [% by | [% by | | T | time | (3) | (6) | (6) |
| Ex. | wt.] | wt.] | wt.] | Catalyst | [° C.] | [min] | [%] | [%] | [%] |
| 6 | 8 | 53 | 39 | Amberlyst 36 | 70 | 22 | 57 | 24 | 42 |
| 7 | 8 | 53 | 39 | Amberlyst 15 | 55 | 36 | 61 | 30 | 49 |
| 8 | 4 | 55 | 41 | Amberlyst 15 | 55 | 11 | 43 | 28 | 65 |
| 9 | 4 | 55 | 41 | Amberlyst 15 | 55 | 48 | 59 | 39 | 66 |

### C) Etherification of 1-methylcyclopentene with methanol

### Example 10

### Preparation of 1-methoxy-1-methylcyclopentane (molar ratio olefin:methanol=1/1.9)

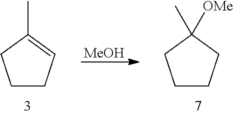

1-Methylcyclopentene (99.2 g, 1.21 mol) and methanol (75.0 g, 2.34 mol) were introduced as initial charge in a 3-neck flask under nitrogen. A Soxhlet attachment with an extraction thimble filled with 15.0 g of Amberlyst 15 was placed on the flask and the mixture was heated to reflux (oil bath: 85° C.). After a total of 67 h, the oil bath was removed and the reaction mixture was cooled. A GC sample was taken, which revealed a conversion of ca. 94%.

Composition of the product mixture:

### MeOH: 19.7%

3 (1-methylcyclopentene): 4.6%
7 (ether): 72.5%

By using a Soxhlet, the following effect is achieved if the temperature is adjusted to 85° C.: the two starting materials methylcyclopentene and methanol boil and then condense at the reflux condenser. The condensed mixture then passes into the reaction zone with the ion exchanger and reacts to give the product 1-methoxy-1-methylcyclopentane. Since its the boiling temperature is above 85° C., the product can be enriched in the bottom to virtually complete conversion.

### Example 11

### Preparation of 1-methoxy-1-methylcyclopentane (olefin:MeOH=1/5.2)

Procedure analogous to Example 10, using

99.0 g (1.20 mol) of 1-methylcyclopentene,
200.0 g (6.24 mol) of methanol and
27.1 g of Amberlyst 15. The reaction time was 18.5 h.

A GC sample showed a conversion of 96%.

Composition of the product mixture:

### MeOH: 35.5%

3 (1-methylcyclopentene): 2.7%
7 (ether): 60.1%

### Example 12

### Preparation of 1-methoxy-1-methylcyclopentane (olefin:MeOH=1/5.1; short reaction time)

Procedure analogous to Example 11, using

100.0 g (1.22 mol) of 1-methylcyclopentene,
200.0 g (6.24 mol) of methanol and
27.1 g of Amberlyst 15. The reaction time was 4.6 h.

A GC sample showed a conversion of 96%.

Composition of the product mixture:

### MeOH: 33.7%

3 (1-methylcyclopentene): 2.3%
7 (ether): 61.7%

In this example, it is shown that a good conversion/yield is achieved even with reasonable residence times.

### Example 13

### Preparation of 1-methoxy-1-methylcyclopentane (without Soxhlet)

1-Methylcyclopentene (50.0 g, 0.61 mol), methanol (200.0 g, 6.24 mol) and 25.0 g of Amberlyst 15 were introduced as initial charge in a 3-neck flask under nitrogen. The mixture was heated to 50° C. After 6 h, a GC sample was taken which showed a conversion of ca. 50%. After 24 h, a further GC sample was taken which showed a conversion of ca. 67%. Neither a longer reaction time, nor the addition of 5.0 g of Amberlyst 15 led to a further conversion.

### Example 14

### Preparation of 1-methoxy-1-methylcyclopentane starting from a mixture which comprises components 2, 3, 4 and 5

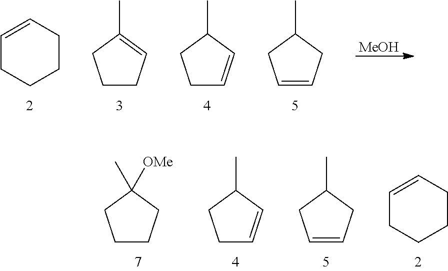

The experiment was carried out analogously to Example 12 with Amberlyst 15 as catalyst. The following starting material mixture was used:

MeOH (31 GC area %), 2 (14 GC area %), 3 (38 GC area %), 4 (10 GC area %), 5 (4 GC area %). After a reaction time of 19 h at 48° C., the following mixture was obtained: MeOH (28 GC area %), 2 (14 GC area %), 3 (15 GC area %), 4 (10 GC area %), 5 (4 GC area %), 7 (25 GC area %).
The example shows that only compound 3 reacts to give the product 7. According to GC analysis, compounds 2, 4 and 5 do not react and can be returned to the gas-phase reaction (see Examples 1-3).

### Example 15

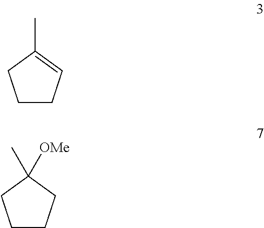

A mixture consisting of compounds 3 and 7 was subjected to fractional distillation at a pressure between 1 bar and 100 mbar. The apparatus used was a 1 liter flask with a 20 cm column with 3 mm V2A Raschig rings.

| | | |
|---|---|---|
| Compounds | Boiling | |
| [in GC area %] | point | Pressure |

| Sample | g | 3 | 7 | [° C.] | bar |
|---|---|---|---|---|---|
| Feed | 431.0 | 20.4 | 73.9 | | |
| Fr. 1 | 40.7 | 96.0 | 0.9 | 22-35 | 1-0.1 |
| Fr. 2 | 34.3 | 79.2 | 19.3 | 35-61 | 1-0.1 |
| Fr. 3 | 23.0 | 33.7 | 65.9 | 47-70 | 1-0.1 |
| Fr. 4 | 10.4 | 8.5 | 91.2 | 70-71 | 0.1 |
| Fr. 5 | 71.6 | 1.2 | 98.6 | 72 | 0.1 |
| Fr. 6 | 183.6 | 0.0 | 99.6 | 72 | 0.1 |
| Fr. 7 | 27.6 | 0.0 | 99.5 | 72 | 0.1 |
| Bottom | 24.1 | | | | |

The experiment shows the distillative separation of 3 and 7.

1. A process for the preparation of 1-methylcyclopentene by thermal reaction of cyclohexanol or cyclohexene or mixtures of both compounds to give 1-methylcyclopentene, wherein the resulting by-products 3-methylcyclopentene and 4-methylcyclopentene (double-bond isomers of 1-methylcyclopentene) are returned to the reaction.

2. The process according to claim 1, wherein unreacted cyclohexene is additionally returned to the reaction.

3. The process according to claim 1 or 2, which is carried out continuously.

4. A process for the preparation of cyclopentane derivatives of the following formula I 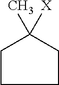
in which X is a hydroxyl group, an alkoxy group or a chlorine atom, by thermal reaction of cyclohexanol or cyclohexene to give 1-methylcyclopentene (1^{st} stage) and subsequent addition of compounds HX, where X has the above meaning, onto the double bond of the 1-methylcyclopentene (2^{nd} stage), wherein
the resulting by-products 3-methylcyclopentene and 4-methylcyclopentene from the product mixture of the 1^{st} or 2^{nd} stage are returned to the reaction in the 1^{st} stage.

5. The process according to claim 4, wherein X in formula I is a hydroxyl group or a C1- to C10-alkoxy group, and HX is correspondingly H₂O or a C1- to C10-alkanol.

6. The process according to any one of claim 4 or 5, wherein X in formula I is a C1- to C10-alkoxy group, and in the 2^{nd} stage the addition reaction of the C1- to C10 alcohol onto the 1-methylcyclopentene takes place by means of a reactive distillation.

7. The process according to any one of claims 4 to 6, wherein the process is carried out continuously.

8. The process according to any one of claims 4 to 7, wherein the resulting by-products 3-methylcyclopentene and 4-methylcyclopentene from the product mixture in the 1^{st} stage are returned to the reaction in the 1^{st} stage.

9. The process according to claim 8, wherein 1-methylcyclopentene and optionally water are separated off from the product mixture from the 1^{st} stage, and 3-methylcyclopentene and 4-methylcyclopentene and unreacted cyclohexene are returned to the reaction in the 1^{st} stage.

10. The process according to any one of claim 8 or 9, wherein unreacted 1-methylcyclopentene and HX from the product mixture in the 2^{nd} stage are returned to the reaction in the 2^{nd} stage.

11. The process according to any one of claims 4 to 7, wherein
after the 1^{st} stage, no separation off and recycling of 3-methylcyclopentene and 4-methylcyclopentene takes place and
3-methylcyclopentene and 4-methylcyclopentene from the product mixture of the 2^{nd} stage are returned to the reaction in the 1^{st} stage.

12. The process according to claim 11, wherein product mixture from the 2^{nd} stage comprises, besides the desired cyclopentane derivative of the formula I, the following compounds:
unreacted 1-methylcyclopentene
the by-products 3-methylcyclopentene and 4-methylcyclopentene
unreacted cyclohexene
optionally water.

13. The process according to claim 11 or 12, wherein, in the case X═OH, after carrying out the 1^{st} stage, no separation off of water takes place.

14. The process according to any one of claims 11 to 13, wherein, in the case X═OH, after the 1^{st} stage, 1 to 100 mol of water per 1 mol of 1-methylcyclopentene are added.

15. The process according to any one of claims 11 to 14, wherein the desired cyclopentane derivative and optionally water are separated off from the product mixture from the 2^{nd} stage, and 3-methylcyclopentene and 4-methylcyclopentene as well as unreacted cyclohexene and 1-methylcyclopentene are returned to the reaction in the 1^{st} stage.
